(19) United States
(12) Patent Application Publication
(10) Pub. No.: US 2025/0098954 A1
(43) Pub. Date: Mar. 27, 2025

(54) MEDICAL DILATOR WITH ECHOGENETIC MARKER

(71) Applicant: BioProtect Ltd., Tzur Yigal (IL)

(72) Inventors: Gil Rosen, Harutzim (IL); Shaul Shohat, Kfar HaOranim (IL); Shahar Miller, Givat Ada (IL)

(21) Appl. No.: 18/726,203

(22) PCT Filed: Jan. 24, 2023
(86) PCT No.: PCT/IB2023/050604

§ 371 Date: Jul. 2, 2024

Related U.S. Application Data
(60) Provisional application No. 63/302,581, filed on Jan. 25, 2022.

(51) Int. Cl. A61B 1/32; A61B 8/12; A61B 17/3203; A61B 90/00; A61M 29/00

(52) U.S. Cl. CPC A61B 1/32; A61B 8/12; A61B 17/3203; A61B 90/39; A61M 29/00; A61B 2090/3925

(57) ABSTRACT

Creating a dissected tissue space in a human subject by using ultrasonic guidance includes inserting a distal end of a. dilator assembly into a tissue space of a. subject, causing and regulating distal movement of the dilator assembly, and subsequent to cessation of the distal movement, deploying an inter-tissue or inter-organ spacer in the tissue space so as to separate a. first tissue from a second tissue, or a. first organ from a second organ. In one example, the dissected tissue space is between a prostate and a rectal wall of a human subject. A medical dilator kit for an ultrasound-guided tissue dissection includes a beveled-tip medical dilator and a dilator-sheath having an internal diameter greater than or equal to an external diameter of the dilator.

### CROSS-REFERENCE TO RELATED APPLICATION

This application claims the benefit of priority to U.S. Provisional Application No. 63/302,581, filed Jan. 25, 2022, which is incorporated by reference herein in its entirety.

### FIELD OF THE INVENTION

The present invention relates to medical dilators for tissue dissection, and particularly to dilators used in ultrasound-guided tissue dissection.

### BACKGROUND

Inter-tissue and inter-organ spacers are often used for creating and occupying a dissected tissue space in human subject. Examples of such spacers can be found in co-pending U.S. patent application Ser. No. 16/273,030, published as US Patent Publication No. US20190239849A1, which is incorporated herewith by reference in its entirety. Typically such spacers are used to distance a healthy tissue or organ from another tissue or organ that is targeted for a treatment such as, for example, a radiation treatment. The tissue dissection process and spacer placement can reduce the exposure of the healthy tissue to the potentially negative effects of the treatment. The spacers are often introduced into the tissue spaces while the surgeon is guided by ultrasound imaging. In many instances the process of locating the spacer at a precise location can be frustrated by the difficulty in distinguishing the correct location for the distal limit of the spacer. An improved approach to positioning the spacer is needed to make the ultrasound-guided tissue-dissection process easier and more accurate.

### SUMMARY

A method is disclosed, according to embodiments of the invention, for creating a dissected tissue space in a human subject by using ultrasonic guidance. The method comprises: (a) inserting a distal end of a dilator assembly, i.e., a sheathed dilator, into a tissue space of a subject, the dilator assembly comprising: (i) a medical dilator comprising a beveled distal section and an elongate cylindrical section proximally abutting the beveled section, and (ii) a dilator-sheath arranged to coaxially surround at least a portion of the cylindrical section of the dilator, the dilator-sheath comprising a hyperechoic marker having an acoustic impedance at least twice that of an outer cylindrical surface portion of the dilator-sheath adjacent to the marker, at least a portion of the hyperechoic marker being proximally displaced from a distal end of the dilator-sheath and distally displaced from a proximal end of the dilator-sheath. The method further comprises: (b) causing and regulating distal movement of the dilator assembly, the causing and regulating including: responsively to receiving an ultrasound image indicating that the hyperechoic marker has reached a target marker-location in the tissue space, ceasing the distal movement; and (c) subsequent to the cessation of the distal movement, deploying an inter-tissue or inter-organ spacer in the tissue space so as to separate a first tissue from a second tissue, or a first organ from a second organ, wherein at least a portion of the spacer is deployed beyond the target marker-location.

In some embodiments, substantially all of the spacer can be deployed beyond the target marker-location.

In some embodiments, a distance from the hyperechoic marker to the distal end of the dilator-sheath can match a length of the deployed spacer. In some such embodiments, the distance can be from an edge of the hyperechoic marker or from another echogenically distinguishable portion thereof.

In some embodiments, the first organ can be the subject's prostate, and the second organ can be the subject's rectum. In some embodiments, the first organ can be the subject's cervix, and the second organ can be the subject's rectum. In some such embodiments, the inserting can be through an incision in the subject's perineum, and/or the ultrasound image can be received from a transrectal ultrasound probe.

In some embodiments, the first organ can be the subject's liver, pancreas or head of the pancreas, and the second organ can be the subject's duodenum or small intestine. In some such embodiments, the inserting can be through an incision in the subject's abdominal wall, and the ultrasound image is received from an endoscopic ultrasound probe.

In some embodiments, the spacer can include a gel.

In some embodiments, the spacer can include a balloon. In some such embodiments, the deploying of the inter-tissue or inter-organ spacer in the tissue space can include (i) withdrawing the dilator, (ii) inserting a deflated tissue-dissection balloon through an interior lumen of the dilator-sheath until a distal end of the deflated balloon is aligned with a distal end of the sheath, and/or (iii) withdrawing the dilator-sheath to expose the balloon, and (iv) inflating the balloon.

In some embodiments, a distal-tip section of the dilator-sheath can include a beveled circumference having a beveling angle greater than a slant angle of the beveled distal section of the dilator.

In some embodiments, the at least a portion of the hyperechoic marker can be displaced at least 2 cm and not more than 8 cm from the distal end of the dilator-sheath. In some embodiments, the at least a portion of the hyperechoic marker can be displaced at least 3 cm and not more than 6 cm from the distal end of the dilator-sheath. In some embodiments, the at least a portion of the hyperechoic marker can be displaced at least 4 cm and not more than 5 cm from the distal end of the dilator-sheath.

In some embodiments, during the inserting, the dilator assembly can be arranged to surround a portion of a guide needle. In some such embodiments, the causing and regulating distal movement can include withdrawing the needle before the hyperechoic marker has reached a target marker-location

In some embodiments, during the inserting, the dilator assembly can be arranged to surround a portion of a guidewire. In some such embodiments, the causing and regulating distal movement can include withdrawing the guidewire before the hyperechoic marker has reached a target marker-location.

In some embodiments, the method can additionally include hydro-dissecting the first tissue from the second tissue, or the first organ from the second organ, before the hyperechoic marker has reached a target marker-location.

In some embodiments, the hyperechoic marker can comprise a metal or metal alloy, and the outer cylindrical surface portion of the dilator-sheath comprises a polymer.

In some embodiments, the hyperechoic marker can comprise a surface feature on the outer cylindrical surface portion of the dilator-sheath formed by an action selected from a group of actions that includes etching, engraving, cutting, chipping, rubbing, filing, scraping, rasping and abrading.

In some embodiments, the hyperechoic marker can have an acoustic impedance at least 5 times, or at least 10 times, that of an outer cylindrical surface portion of the dilator-sheath adjacent to the marker.

A method is disclosed, according to embodiments, for creating a dissected tissue space between a prostate and a rectal wall of a human subject, the method comprising: (a) inserting, through an incision in the subject's perineum, a distal end of a dilator assembly, the dilator assembly arranged to surround a portion of a guiding element penetrating the perineum, the guiding element being one of a guidewire and a guide-needle, the dilator assembly comprising: (i) a distally-beveled dilator, and (ii) a sheath arranged to coaxially surround a portion of the dilator that excludes a distal tip of the dilator, the sheath comprising a hyperechoic marker having an acoustic impedance at least twice that of an outer cylindrical surface portion of the sheath adjacent to the marker, at least a portion of the hyperechoic marker being disposed between 4 and 5 cm from a distal end of the sheath. The method additionally comprises: (b) causing and regulating distal movement of the dilator assembly, the causing and regulating including: (i) responsively to the distal tip of the dilator reaching a first surface of the rectal wall, withdrawing the guiding element, and (ii) responsively to receiving an image from a transrectal ultrasound probe indicating that the hyperechoic marker is parallel to an apex of the prostate, ceasing the distal movement. The method additionally comprises: (c) subsequent to the cessation of the distal movement and while the hyperechoic marker is parallel to the apex of the prostate, (i) withdrawing the dilator, (ii) inserting a deflated tissue-dissection balloon through an interior lumen of the sheath until a distal end of the deflated balloon is aligned with a distal end of the sheath, and (iii) withdrawing the sheath to expose the balloon; and (d) inflating the balloon to cause thereby a separation of the prostate from the rectal wall.

In some embodiments, it can be that substantially all of the balloon is deployed beyond the target marker-location.

In some embodiments, a distance from the hyperechoic marker to the distal end of the dilator-sheath can match a length of the balloon. In some such embodiments, the distance can be from an edge of the hyperechoic marker or from another echogenically distinguishable portion thereof.

In some embodiments, the method can additionally include hydro-dissecting the first tissue from the second tissue, or the first organ from the second organ, before the hyperechoic marker has reached a target marker-location.

In some embodiments, the hyperechoic marker can comprise a metal or metal alloy, and the outer cylindrical surface portion of the dilator-sheath comprises a polymer.

In some embodiments, the hyperechoic marker can comprise a surface feature on the outer cylindrical surface portion of the dilator-sheath formed by an action selected from a group of actions that includes etching, engraving, cutting, chipping, rubbing, filing, scraping, rasping and abrading.

In some embodiments, the hyperechoic marker can have an acoustic impedance at least 5 times, or at least 10 times, that of an outer cylindrical surface portion of the dilator-sheath adjacent to the marker.

In some embodiments, a distal-tip section of the dilator-sheath can include a beveled circumference having a beveling angle greater than a slant angle of the beveled distal section of the dilator.

According to embodiments, a medical dilator kit for an ultrasound-guided tissue dissection comprises: (a) a beveled-tip medical dilator comprising (i) a conical frustum section formed in a distal portion of the dilator, (ii) a cylinder section formed proximally to the conical frustum portion to abut the base thereof; and (b) dilator-sheath having an internal diameter greater than or equal to an external diameter of the dilator, the sheath comprising a hyperechoic marker having an acoustic impedance at least twice that of an outer cylindrical surface portion of the dilator-sheath adjacent to the marker, at least a portion of the hyperechoic marker being proximally displaced from a distal end of the dilator-sheath and distally displaced from a proximal end of the dilator-sheath.

In some embodiments, the at least a portion of the hyperechoic marker can be displaced at least 3 cm and not more than 6 cm from the distal end of the dilator-sheath. In some embodiments, the at least a portion of the hyperechoic marker is displaced at least 4 cm and not more than 5 cm from the distal end of the dilator-sheath.

In some embodiments, a distal-tip section of the dilator-sheath can include a beveled circumference. In some embodiments, a beveling angle of the distal-tip section of the dilator-sheath can be greater than a slant angle of the conical frustrum section of the dilator.

In some embodiments, the hyperechoic marker can be attached to the outer cylindrical surface portion of the dilator-sheath. In some embodiments, the hyperechoic marker can be formed in the outer cylindrical surface portion of the dilator-sheath.

In some embodiments, the dilator can include a longitudinal channel surrounding a central axis of the dilator and interiorly traversing the combined lengths of the conical frustum and cylinder sections, the longitudinal channel having an internal diameter greater than or equal to 1 mm and less than or equal to 2 mm.

In some embodiments, the dilator and dilator-sheath can be in an assembled state in which the dilator-sheath is arranged to coaxially surround at least a portion of the cylindrical section of the dilator.

In some embodiments, the dilator kit can additionally comprise an inter-tissue or inter-organ spacer. In some such embodiments, the spacer can include a balloon. In some embodiments, the location of the hyperechoic marker on the outer cylindrical surface portion of the dilator-sheath can correspond to a length of the spacer. In some embodiments, the location of the hyperechoic marker on the outer cylindrical surface portion of the dilator-sheath cam be equal to a length of the spacer.

In some embodiments, the dilator kit can additionally comprise a guide-needle. In some embodiments, the dilator kit can additionally comprise a guidewire. In some embodiments, the dilator kit can additionally comprise one or more interiorly sterile containers for housing the other elements of the dilator kit.

In some embodiments, the hyperechoic marker can comprise a metal or metal alloy, and the outer cylindrical surface portion of the dilator-sheath comprises a polymer. In some embodiments, the hyperechoic marker can comprise a surface feature on the outer cylindrical surface portion of the dilator-sheath formed by an action selected from a group of actions that includes etching, engraving, cutting, chipping, rubbing, filing, scraping, rasping and abrading.

### BRIEF DESCRIPTION OF THE DRAWINGS

Embodiments of invention will now be described further, by way of example, with reference to the accompanying drawings, in which the dimensions of components and features shown in the figures are chosen for convenience and clarity of presentation and not necessarily to scale. In the drawings:

FIG. 1 is a schematic side-rear perspective view of a dilator assembly according to embodiments of the present invention.

FIGS. 2A and 2B are schematic illustrations of a dilator-sheath bearing a hyperechoic marker according to embodiments of the present invention.

FIGS. 3A, 3B and 3C are schematic cross-sectional views of a dilator according to embodiments of the present invention.

FIGS. 4A and 4B are schematic cross-sectional views of a dilator-sheath according to embodiments of the present invention.

FIG. 5 is a schematic cross-sectional view of a dilator assembly comprising the dilator of FIG. 3A and the dilator-sheath of FIG. 4A, according to embodiments of the present invention.

FIGS. 6A through 6F are block diagrams of kits comprising dilator assemblies and/or other components for use in dissecting between tissues or between organs using ultrasonic guidance, according to embodiments of the present invention.

FIGS. 7, 8 and 9 show flowcharts of methods for dissecting between tissues or between organs using ultrasonic guidance, according to embodiments of the present invention.

FIGS. 10A through 10G illustrate application of the methods of FIGS. 7, 8 and 9 to an exemplary use case, according to embodiments of the present invention.

### DETAILED DESCRIPTION OF THE ILLUSTRATED EMBODIMENTS

Embodiments of the invention are herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Throughout the drawings, like-referenced characters are generally used to designate like elements.

For convenience, in the context of the description herein, various terms are presented here. To the extent that definitions are provided, explicitly or implicitly, here or elsewhere in this application, such definitions are understood to be consistent with the usage of the defined terms by those of skill in the pertinent art(s). Furthermore, such definitions are to be construed in the broadest possible sense consistent with such usage.

Apparatuses and methods for dissection of one tissue from another, or one organ from another, are disclosed. The dissection can be accomplished by delivering a spacer, such as an inflatable balloon or an appropriate gel, to reside between the tissues or organs until such time that the spacer biodegrades and/or is removed. Such spacers are known to be useful in cases where physical separation between adjacent tissues and/or organs is desirable, for example, to protect one tissue or organ from effects of a treatment to the second tissue or organ-for example, a radiation treatment. In embodiments, an apparatus for use in the dissection process includes a dilator assembly configured to facilitate locating the spacer at a desired or optimal location, and to facilitate the insertion of the spacer at the desired or optimal location. A dilator assembly can be inserted into a subject's body, for example through an incision in the subject's perineum or abdominal wall, depending on which tissues or organs are involved.

The dilator assembly can be inserted along (i.e., surrounding, e.g., concentrically surrounding) a guide needle or guidewire that interiorly traverses the dilator assembly. The dilator assembly preferably comprises a dilator and a dilator-sheath mounted concentrically around the dilator. Once located at a target location, the dilator can be removed from within the sheath, for example by withdrawing the dilator proximally, such that the spacer can be passed through the interior of the sheath and deployed at or beyond the distal end of the sheath. In embodiments, the locating of the spacer at the desired or optimal target location can be better accomplished by adding a hyperechoic marker to an external surface of the dilator assembly, the marker being formed so as to be easily seen by a surgeon, e.g., on a monitor or other imaging device in communication with an ultrasound probe. The materials and shape of the hyperechoic marker can be selected to be more easily ‘seen’ in sufficiently high contrast to the surrounding surface of the dilator assembly adjacent to the marker, e.g., by ensuring that the acoustic impedance of the marker is at least 5 times, or at least 10 times, the acoustic impedance of the surface area of the sheath near the marker or next to the marker. For example, a metal or metal-alloy marker can be suitable for use with a sheath formed of a polymeric material such as, in a non-limiting example, a polypropylene. Additionally or alternatively, a marker can be formed in the polymeric material of the sheath itself, for example by etching. engraving, cutting, chipping, rubbing, filing, scraping, rasping, abrading or any equivalent process of creating ultrasound-reflective edges and/or angled surfaces in the material.

With a higher acoustic impedance than the surrounding sheath surface area, the marker can be helpful in accurately locating the spacer at a desired or optimal location. In exemplary use of the apparatus, a target marker-location is defined at a known distance from the desired/optimal target deployment-location selected for deployment of the spacer (the deployment of the spacer being from the distal end of the sheath). The known distance between target deployment-location and target marker-location is used to define a location, i.e., a distance from the distal end of the sheath, of a discernible (e.g., sufficiently visible in a ultrasound image) portion of the marker—e.g., a leading or trailing edge of the marker. Thus when the specified portion of the marker reaches the target marker-location, the surgeon can confidently infer that the distal end of the sheath—from which the spacer is to be deployed—is located at the desired or optimal deployment-location for deployment of the spacer. The sheath (with marker) and foregoing process can be particularly useful in surgical implementations in which a target marker-location is more visible in ultrasound imaging and/or more readily identifiable on an ultrasound monitor than is the target deployment-location.

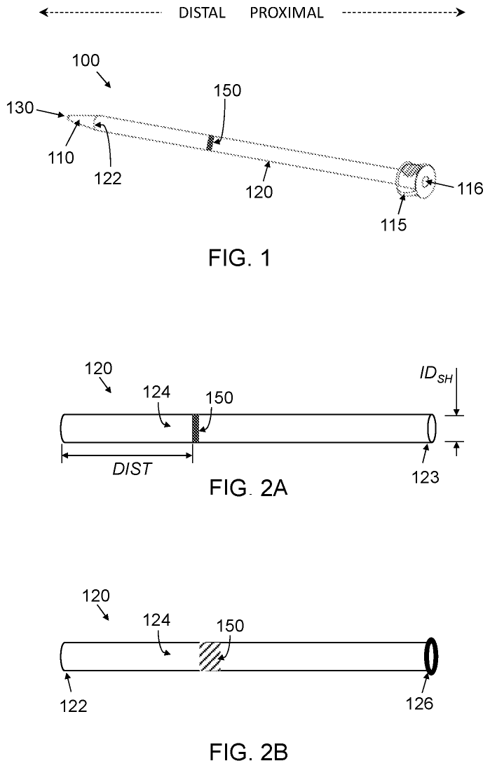

Referring now to the figures and in particular to FIG. 1, an exemplary assembled sheathed dilator 100 according to embodiments is schematically illustrated in a rear-side perspective view. Note: the terms ‘sheathed dilator’ and ‘dilator assembly’ are used interchangeably and equivalently here. The sheathed dilator 100 of FIG. 1 includes a medical dilator 110 partly surrounded by a sheath 120 such that only a distal portion 117 of the dilator 110 and a proximal dilator knob 115 are visible. The ‘surrounding’ is along a longitudinal axis of the dilator assembly 100 and is preferably concentric. The terms ‘distal’ and ‘proximal’ as used throughout this disclosure and in the claims appended thereto are to be understood according their accepted usage, wherein the ‘distal’ direction is the direction further into a patient's body and away from a user, e.g., a medical practitioner using the device, while the proximal direction indicates the opposite direction. Distal and proximal directions are shown for clarity in FIG. 1; a convention of distal being to the left of the page, and proximal being to the right of the page, is maintained throughout the figures.

Reference is made to FIGS. 2A and 2B. The sheath 120 includes a hyperechoic marker 150 around at least a portion of the circumference of the sheath 120. The at least a portion of the circumference can be continuous or non-continuous, and must be substantial enough so that the marker 150 is adequately visible in ultrasound imaging for the purpose. In embodiments, at least a portion of the marker 150, e.g., a distal edge of the marker 150, a proximal edge of the marker 150, or a point therebetween, e.g., an echogenically discernible point, is at least 2 cm and not more than 10 cm from the distal end 122 of the dilator-sheath 120. In embodiments the distance (indicated by the arrow marked DIST in FIG. 2A) between the at least a portion of the marker 150 and the distal end 122 of the sheath 120 can be set in accordance with the nature and/or dimensions of the specific tissues and/or organs involved, given that the goal of setting the distance DIST is to match the distance between a target deployment-location of the spacer and an ultrasound-visible marker-location. In embodiments, the distance between the at least a portion of the marker 150 and the distal end 122 of the sheath 120 can be 2-9 cm, or 2-8 cm, or 2-7 cm, or 2-6 cm, or 2-5 cm, or 2-4 cm, or 2-3 cm, or 3-10 cm, or 3-9 cm, or 3-8 cm, or 3-7 cm, or 3-6 cm, or 3-5 cm, or 3-4 cm, or 4-10 cm, or 4-9 cm, or 4-8 cm, or 4-7 cm, or 4-6 cm, or 4-5 cm, or 5-10 cm, or 5-9 cm, or 5-8 cm, or 5-7 cm, or 5-6 cm, or 6-10 cm, or 6-9 cm, or 6-8 cm, or 6-7 cm, or 7-10 cm, or 7-9 cm, or 7-8 cm, or 8-10 cm, or 8-9 cm, or 9-10 cm, each of the ranges being inclusive. An interior diameter of the sheath 120, indicated in FIG. 2A by the arrows ID_{SH}, can be at least 2 mm and is typically not larger than 10 mm unless required for a spacer that is particularly large even when deflated and folded or rolled up into a tube. Exemplary values of ID_{SH} are between 4 and 8 mm, between 5 and 7 mm, between 5 and 6 mm, between 5.5 and 6.5 mm, or between 6 and 7 mm, each of the ranges being inclusive. The marker 150 is distally displaced from the proximal end 123 of the sheath 120; in some embodiments (not illustrated) a marker 150 can extend all the way to the proximal end 123 of the sheath 120, and in such cases the leading edge of the marker 150 can still be usable for the purposes laid out hereinabove.

The respective markers 150 of FIGS. 2A and 2B are shown to have different widths (i.e., different fractions of the overall length of the sheath 120) and to have been formed differently. As discussed hereinabove, a hyperechoic marker 150 can be formed by adding material—e.g., a material comprising a metal or metal allow as illustrated in FIG. 2A—to the sheath or by forming the marker 150 in the material of the sheath 120 itself as shown in FIG. 2B, by scoring, gouging, filing, chipping or any other suitable method for marking the surface so as to achieve an adequate degree of ultrasound contrast and visibility. As shown in FIG. 2B, a sheath can include a proximal sheath flange 126. e.g. for easier handling.

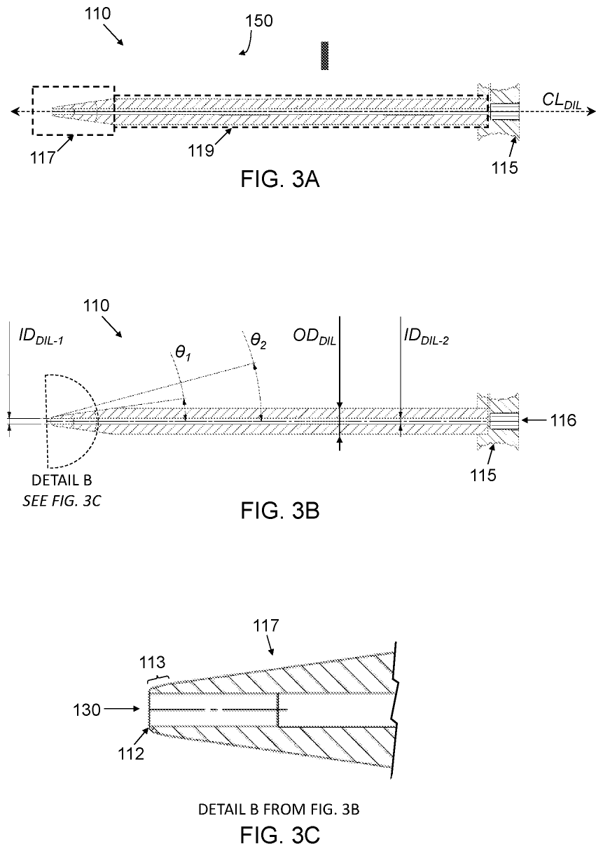

An exemplary dilator 110 is shown in cross-sectional view in FIGS. 3A-3C. The dilator 110 includes a distal conical frustum 117 and, proximal thereto, a cylindrical section 119. A proximal knob 115 can be attached proximally to the cylindrical section 119 and optionally textured to facilitate the rotation of dilator 110 in the hand of a user, e.g., a surgeon. In some embodiments, the conical frustum section 117 and the cylinder section 119 are formed as a single piece. In some embodiments, the conical frustum section 117, the cylinder section 119 and the knob are formed as a single piece. In some embodiments, a dilator 110 can comprise separate pieces joined to form the dilator 110. The dilator 110 (including any and all of its components) should have a central bore traversing the length of the dilator 110 from a proximal opening 130 to a distal opening 116. The central bore is to be suitably sized for a guidewire or guide needle to interiorly traverse the dilator 110 and can have an internal diameter (indicated by arrows ID_{DIL-1} and ID_{DIL-2} in FIG. 3B) of between 0.5 and 3 mm, or between 0.5 and 2.5 mm, or between 0.5 and 2 mm, or between 0.5 and 1.5 mm, or between 1 and 3 mm, or between 1 and 2.5 mm, or between 1 and 2 mm, or between 1.5 and 3 mm, or between 1.5 and 2.5 mm, each of the ranges being inclusive. In some embodiments, the internal diameters ID_{DIL-1} and ID_{DIL-2} are constant throughout the length of the dilator 110: in some embodiments, the internal diameters ID_{DIL-1} and ID_{DIL-2} can vary throughout the length of the dilator 110; and in some embodiments, the internal diameters ID_{DIL-1} and ID_{DIL-2} are constant throughout the length of the dilator 110 but have different tolerances, e.g., wider tolerances at ID_{DIL-2} and narrower tolerances at ID_{DIL-1}. The outer diameter of the cylindrical section 119 of the dilator 110 (indicated by the arrows marked OD_{DIL} in FIG. 3B) is selected to be less than or equal to the inner diameter ID_{SH} of the sheath 120. In embodiments, the outer diameter OD_{DIL} of the cylindrical section 119 of the dilator 110 and the inner diameter ID_{SH} of the sheath 120 can be selected such that ID_{SH}-OD_{DIL} is between 0 and 0.5 mm, or between 0 and 1 mm, both ranges inclusive.

As shown schematically in FIGS. 3B and 3C, the dilator 110 can be distally beveled in two beveling-steps. A first and more substantial beveling is defined by a first angle θ₁, as shown in FIG. 3B, between the external surface of the conical frustum section 117 and a centerline of the dilator 110 (indicated in FIG. 3A by arrow CL_{DIL}), which is parallel to the external surface of the cylindrical section 119. A suitable value for angle θ₁ is between 0° and 15°, or between 5° and 10°, both ranges inclusive. A second bevel is defined by a second angle θ2, as also shown in FIG. 3B, between a further-beveled portion 113 (beginning at or near the distal tip 112 of the conical frustum 117) and the centerline of the dilator 110. The further-beveled portion can be curved or straight, for example at a constant angle θ₂ between 10° and 30°, or between 10° and 20°, both ranges inclusive. The second bevel angle θ₂ is by nature greater than the first bevel angle θ₁, i.e., as both bevel angles are relative to the centerline of the dilator 110.

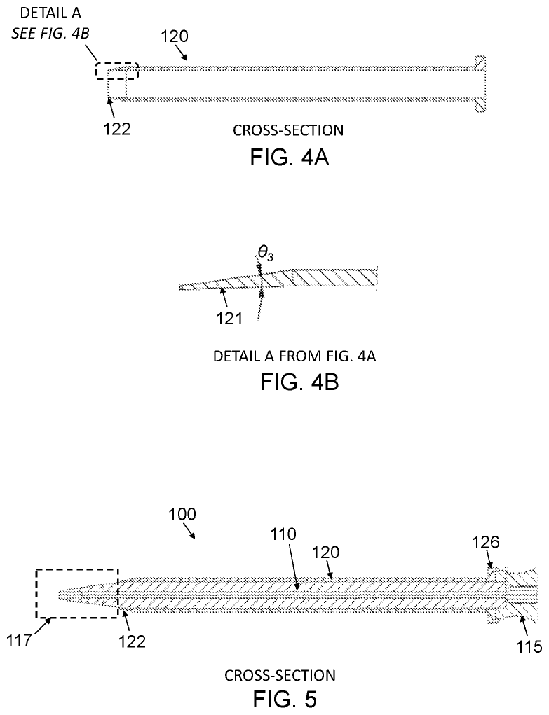

Referring now to FIGS. 4A and 4B, a dilator-sheath 120 is illustrated schematically in cross-section. As per the figures, the distal end 122 of the sheath 120 can be beveled at an angle of θ₃ relative to the interior surface 121 of the sheath 120, which is parallel to centerline CL_{DIL}.

FIG. 5 shows a cross-section of the assembled sheathed dilator 100 comprising the dilator 110 of FIGS. 3A-3C and the dilator sheath 120 of FIGS. 4A-4B. As can be seen, the beveling of the distal end 122 of the sheath 120 serves to transition between the angled surfaces of the conical frustum 117 of the dilator 110 and the straight surface of the continuation of the dilator-sheath 120. In some embodiments, θ₃ can be selected to ‘continue’ the bevel of the dilator, i.e., at angle θ₂ above the horizontal, and in other embodiments, the angle θ₃ is smaller than the angle θ₂ since in any case there is a non-zero step between the conical frustum section 117 of the dilator 110 and the distal end 122 of the sheath 120.

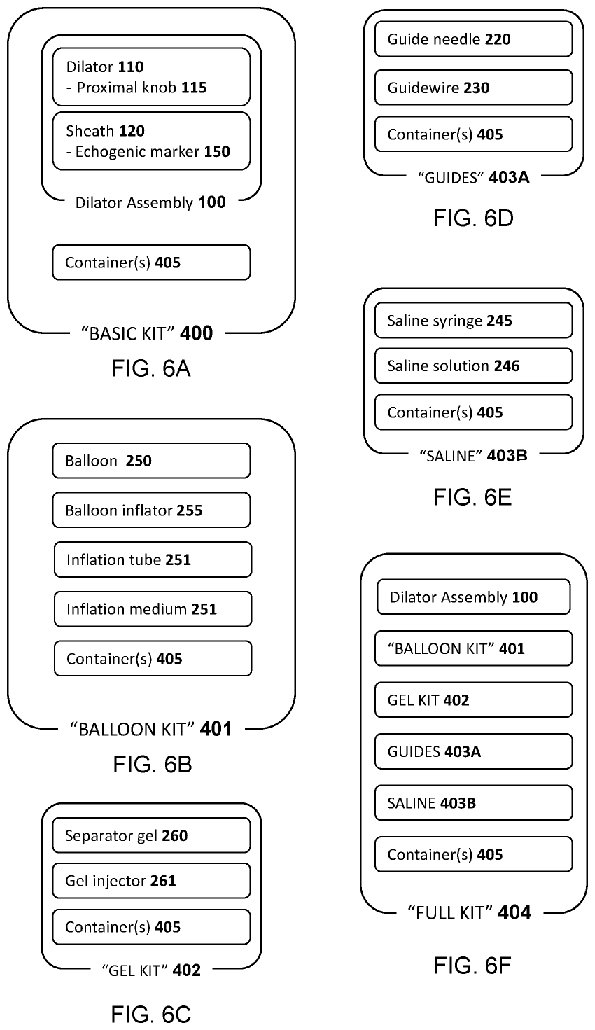

The block diagrams of FIGS. 6A-6F illustrate a variety of kits 400, 401, 402, 403A, 403B, 404 including dilator assemblies 100 and/or related components and apparatus suitable for use in dissecting one tissue from another or one organ from another according to embodiments of the invention. A “basic kit” 400, illustrated in FIG. 6A, includes a dilator assembly 100 according to any of the embodiments disclosed herein, which includes a dilator 110 and, optionally a proximal knob 115 which can be provided separately from the dilator 110, formed as part of the dilator 110, or manufactured as a distinct component but provided when already attached to the dilator 110. The basic kit 400 can also include a container 405 in which the other components are provided. Any container 405 described herein can be an interiorly sterile container of any convenient and appropriate shape for transport and/or storage of the respective kit. A “balloon kit” 401, illustrated in FIG. 6B, includes a number of components related to the insertion and inflation of a spacer balloon 250, as will be described in greater detail hereinbelow in the description of FIGS. 8A-8G. The balloon kit 401 includes the balloon 250, a balloon inflator 251, an inflation tube 251, the inflation medium 251 (e.g., a viscous inflation medium such as a gel), and, optionally, a container 405 in which the other components are provided. As illustrated in FIG. 6C, a “gel” kit 402 includes a separator gel 260 that can be used as a spacer, according to embodiments, instead of a balloon, a gel injector 261, and, optionally, container 405 in which the other components are provided. A “guides” kit 403A, illustrated in FIG. 6D, can include a guide needle 220 and/or a guidewire 230. The guides kit 403A can also include a container 405 in which the other components are provided. As illustrated in FIG. 6E, a “saline” kit can include a syringe 245, a quantity of saline solution 246, and. optionally, container 405 in which the other components are provided. An “advanced kit” 404, as illustrated in FIG. 6F, can include a dilator assembly 100 of the basic kit 4001, components of the balloon kit 401, components of the gel kit 402, components of the guide kit 403A, components of the saline kit 403B, and, optionally one or more containers 405. The skilled artisan will understand that any two or more of the foregoing kits 400, 401, 402, 403A, 403B, 404 and/or any two or more of the constituent components of any of the foregoing kits, can be combined to form a ‘specialty’ kit for use in practicing any of the embodiments disclosed herein. Combined specialty kits can include a single container 405 or multiple containers 405, e.g., a container for each of the kits or components making up the specialty kit. Further, it should be readily understood that not all components of any given kit need be present in order to fall within the scope of embodiments of the invention.

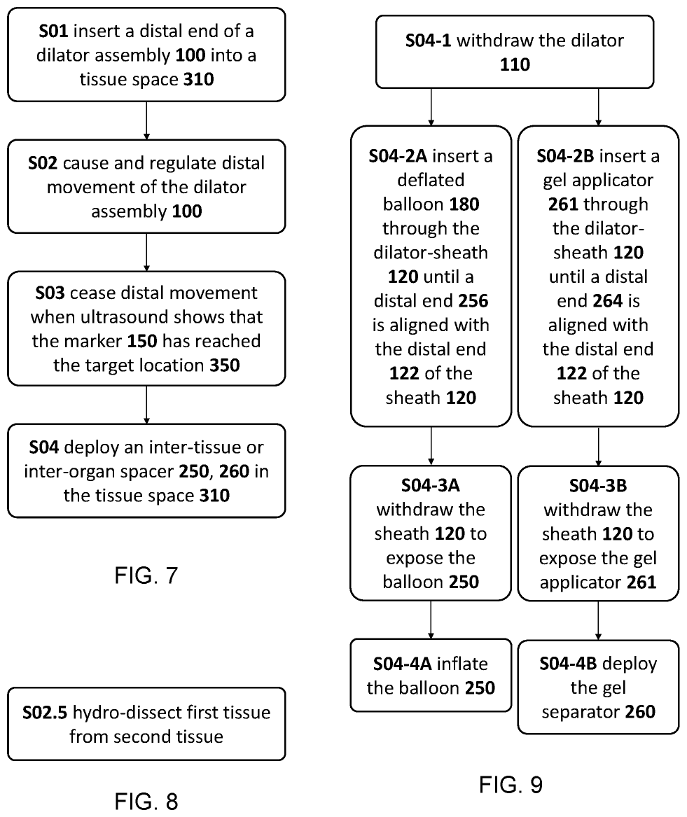

FIG. 7 shows a flowchart outlining steps of a method for creating a dissected tissue space in a human subject by using ultrasonic guidance The method can employ any of the dilator assemblies, dilators and dilator-sheaths disclosed herein, as well as the disclosed hyperechoic markers 150 and other equipment items including, and not exhaustively: balloons 250, balloon inflation tubes 251, balloon inflators 255 and balloon inflation media 257; gel 260 and gel applicators 261; guide needles 220 and guidewires 230; and saline kits 403B and components. The method comprises the following steps:

Step S01 inserting a distal end of a dilator assembly 100 into a tissue space 310 of a subject. The dilator assembly 100 includes a dilator 110 comprising a beveled distal section 117 and an elongate cylindrical section 119 proximally abutting the beveled section 117, and a dilator-sheath 120 arranged to coaxially surround at least a portion of the cylindrical section 119 of the dilator 110. The dilator-sheath 120 comprises a hyperechoic marker 150 having an acoustic impedance at least twice that of an outer cylindrical surface portion 124 of the dilator-sheath 120 adjacent to the marker 150. At least a portion of the hyperechoic marker 150 is proximally displaced from a distal end 112 of the dilator-sheath and distally displaced from a proximal end 123 of the dilator-sheath 120. The distance from the at least a portion of the marker 150 to the distal end 122 of the dilator-sheath 120 can match a length of the spacer 250, 260 once deployed. The distance from the at least a portion of the marker 150 is preferably from an edge of the hyperechoic marker 150 or from another echogenically distinguishable portion. During the inserting the dilator assembly 110 can be arranged to surround a portion of a guide needle 220 or guidewire 230.

Step S02 causing and regulating distal movement of the dilator assembly 100. The causing and regulating of distal movement can include withdrawing the needle 220 (or guidewire 230) before the hyperechoic marker has reached a target marker-location

Step S03 ceasing distal movement of the dilator assembly 100 when an ultrasound image or monitor shows that the marker 150 has reached the target location 350.

Step S04 deploying an inter-tissue spacer or inter-organ spacer (balloon 250 or gel 260) in the tissue space 310 distal to the distal end 122 of the sheath 120. Said deployment is effective to separate a first tissue from a second tissue, or a first organ from a second organ. In a first example, the first organ is the subject's prostate, and the second organ is the subject's rectum. In a second example, the first organ is the subject's cervix, and the second organ is the subject's rectum. In the first two examples, the ‘inserting’ of Step S01 can be through an incision in the subject's perineum, and the ultrasound image can be received from a transrectal ultrasound probe. In a third example, the first organ is the subject's liver or pancreas or head of the pancreas, and the second organ is the subject's duodenum or small intestine. In the third example, the ‘inserting’ of Step S01 can be through an incision in the subject's abdominal wall, and the ultrasound image can be received from an endoscopic ultrasound probe.

The deployment of Step S04 is such that at least a portion of the spacer 250, 260 is deployed beyond the target marker-location 350—but can be such that all, or substantially all (at least 75% or at least 85% or at least 90% or at least 95% or at least 99%) of the spacer 250, 260 is deployed beyond the target marker-location 350.

In some embodiments, the method additionally includes Step S02.5 of FIG. 7: hydrodissecting the first tissue from the second tissue during (e.g., during pauses in) the distal movement of the dilator assembly 100 of Step S02, using, for example components of a saline kit 403B.

In some embodiments, (the deploying an inter-tissue spacer or inter-organ spacer of Step S04 includes the following sub-steps, as illustrated in the flow chart of FIG. 9:

Sub-step S04-1 proximally withdrawing the dilator 110 from within the dilator sheath 120.

If deploying a balloon 250 as spacer:
Sub-step S04-2A inserting a deflated balloon 250 through the dilator-sheath 120 until a distal end 256 of the balloon 250 is aligned with the distal end 122 of the sheath 120.
Sub-step S04-3A withdrawing the sheath 120 to expose the balloon 250
Sub-step S04-4A inflating the balloon 250

Alternatively, uf deploying a gel 260 as spacer:
Sub-step S04-2B inserting a gel applicator 261 through the dilator-sheath 120 until a distal end 264 of the gel applicator 264 is aligned with the distal end 122 of the sheath 120.
Sub-step S04-3B withdrawing the sheath 120 to expose the gel applicator 261
Sub-step S04-4B deploying the gel separator 260

### Exemplary Use Case

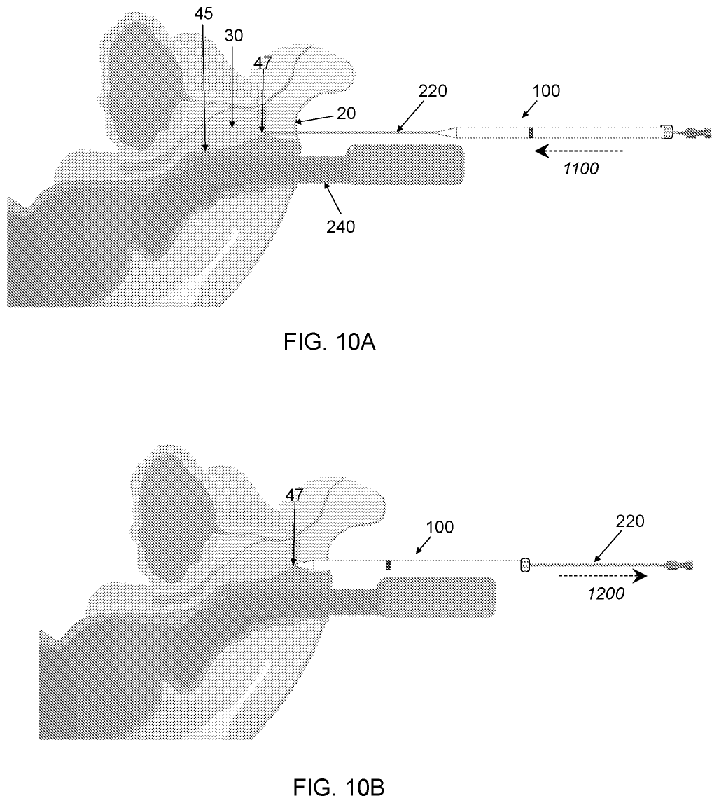

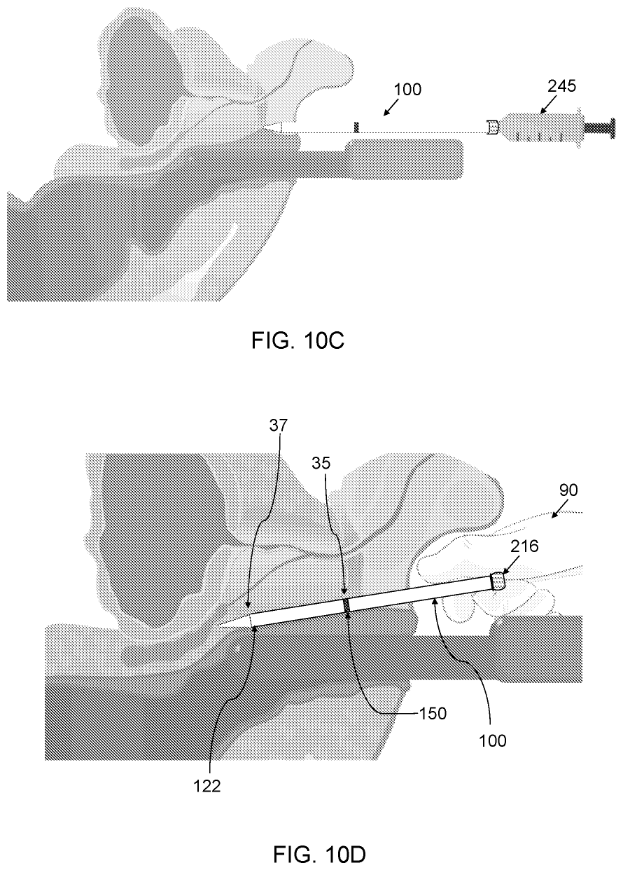

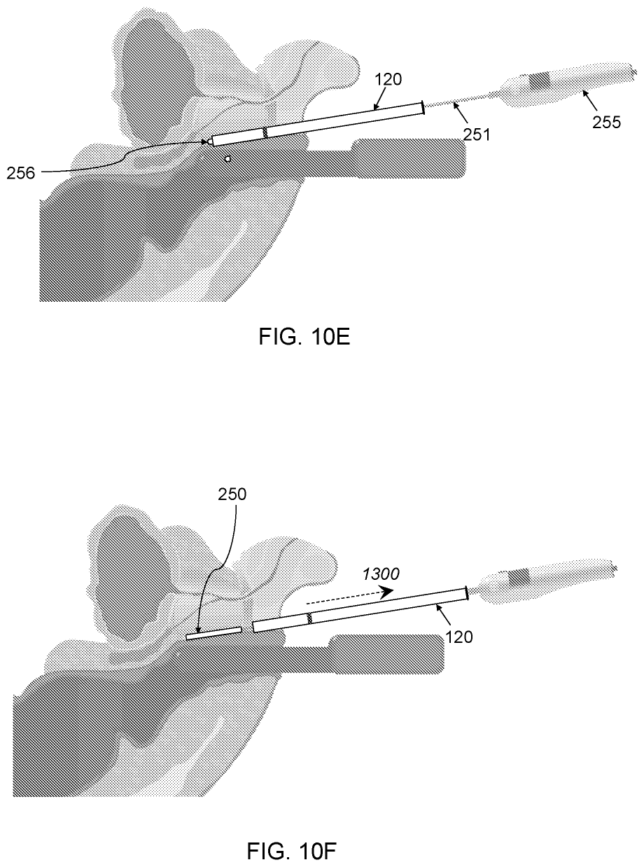

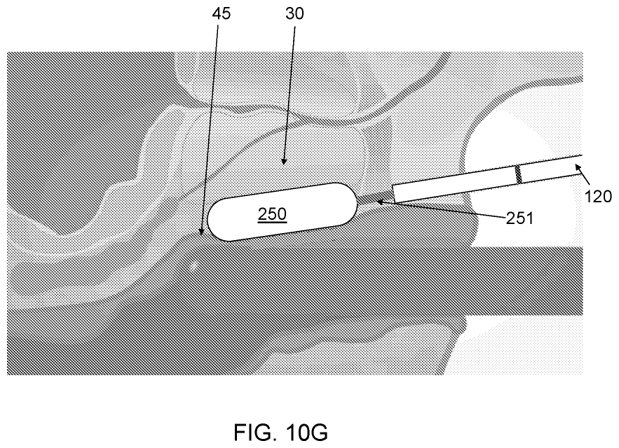

FIGS. 10A-10G illustrate an exemplary use case wherein methods disclosed herein are applied to the dissection of a subject's prostate from the subject's rectum, according to embodiments of the present invention. The exemplary use case illustrates features which are applicable to other examples of tissues and organs as discussed hereinabove, and are not limited to the prostate-rectum case.

As seen in FIG. 10A, a transrectal ultrasound (TRUS) probe 240 has already been deployed for guiding the procedure. A dilator assembly 100 surrounds a guide needle 220, which interiorly traverses the dilator assembly 100, and is advanced in FIG. 10A, in the direction indicated by arrow 1100 over the guide needle 220. The needle has already been advanced to a first surface 47 of the rectal wall 45. In FIG. 10B, the dilator assembly 100 has been advanced along the guide needle 220 and inserted through an incision in the subject's perineum 20 until the distal tip of the dilator component of the dilator assembly 100 reaches said first surface 47 of the rectal wall 45, as in Steps S01 and S02 of FIG. 7. As indicated by arrow 1200, the guide needle 220 is proximally withdrawn once the distal tip of the dilator assembly 100 has reached said first surface 47. As seen in FIG. 10C, after withdrawal of the guide needle 220, a saline syringe 245 has been introduced, a needle (not shown) inserted through the interior lumen of the dilator assembly 100, for hydro-dissection of the tissues (e.g., between the prostate 30 and the rectal wall 45), as per Step S02-5 of FIG. 8, for ease of further distal advancement of the dilator assembly 110.

FIG. 10D shows—after the hydro-dissection, which is optional in some examples—the dilator assembly already advanced, e.g., by the user 90 pushing the dilator assembly 100 and/or turning the dilator knob 216 and/or adjusting the entry angle of the dilator assembly 100, until the hyperechoic marker 150 on the outer surface of the dilator-sheath 120 of the dilator assembly 100 has reached, e.g., become parallel to, the apex 35 of the prostate 30, as per Step S03 of FIG. 7. The apex 35 of the prostate 30, in this exemplary use case, represents the target market-location 350 discussed hereinabove, as applied to the anatomy of the specific use case. The hyperechoic marker 150, e.g., an edge or other echogenically distinguishable portion, is displaced from the distal end 122 of the sheath component 120 of the dilator assembly 120 at a distance (e.g., distance DIST of FIG. 2A) that matches or is based upon a measured or presumed or typical distance from the apex 35 of the prostate 30 to the base 37 of the prostate 30 in the subject. The skilled artisan will understand that placing the hyperechoic marker 150 parallel to the relatively highly ultrasound-visible apex 35 of the prostate 30 can be a more accurate and/or easier task for the surgeon to accomplish than lining up the less ultrasound-visible distal end 122 of the sheath 120 with the less ultrasound-visible base 37 of the prostate 30, while still bringing the distal end 122 of the dilator-sheath 120 to the desired target location for deployment of the spacer.

Referring now to FIG. 10E, the dilator 110 has been withdrawn from the dilator sheath 120 as per Step S04-1 of FIG. 9, and balloon 250 is inserted through the dilator-sheath 120 until a distal end 256 is aligned with the distal end 122 of the sheath 120, as per Step S04-2A of FIG. 9. The balloon is connected to the inflator 255 by inflation tube 251. In FIG. 10F, it can be seen that the dilator sheath 120 is proximally withdrawn, as indicated by arrow 1300 and as per Step S04-3A of FIG. 9, to expose the folded-up or rolled-up uninflated balloon 250. FIG. 10G shows that the balloon 250 has been inflated, as per Step S04-4A of FIG. 9, to a desired thickness between the prostate 30 and the rectal wall 45, using inflator medium (not shown) injected by the balloon inflator 255 through the inflation tube 251. The balloon inflator 255 and the inflation tube 251 can then be withdrawn so as to leave the inflated balloon spacer 250 in place.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. It will be appreciated by persons skilled in the art that the present invention is not limited to what has been particularly shown and described hereinabove. Rather, the scope of the present invention includes both combinations and sub-combinations of the various features described hereinabove, as well as variations and modifications thereof that are not in the prior art, which would occur to persons skilled in the art upon reading the foregoing description.

1. A method for creating a dissected tissue space in a human subject by using ultrasonic guidance, the method comprising:
a. inserting a distal end of a dilator assembly into a tissue space of a subject, the dilator assembly comprising:
i. a medical dilator comprising a beveled distal section and an elongate cylindrical section proximally abutting the beveled section, and
ii. a dilator-sheath arranged to coaxially surround at least a portion of the cylindrical section of the dilator, the dilator-sheath comprising a hyperechoic marker having an acoustic impedance at least twice that of an outer cylindrical surface portion of the dilator-sheath adjacent to the marker, at least a portion of the hyperechoic marker being proximally displaced from a distal end of the dilator-sheath and distally displaced from a proximal end of the dilator-sheath;
b. causing and regulating distal movement of the dilator assembly, the causing and regulating including:
responsively to receiving an ultrasound image indicating that the hyperechoic marker has reached a target marker-location in the tissue space, ceasing the distal movement; and
c. subsequent to the cessation of the distal movement, deploying an inter-tissue or inter-organ spacer in the tissue space so as to separate a first tissue from a second tissue, or a first organ from a second organ, wherein at least a portion of the spacer is deployed beyond the target marker-location.

2. The method of claim 1, wherein substantially all of the spacer is deployed beyond the target marker-location.

3. The method of claim 1, wherein a distance from the hyperechoic marker to the distal end of the dilator-sheath matches a length of the deployed spacer.

4. (canceled)

5. The method of claim 1, wherein the first organ is the subject's prostate, and the second organ is the subject's rectum.

6. The method of claim 1, wherein the first organ is the subject's cervix, and the second organ is the subject's rectum.

7. (canceled)

8. The method of claim 1, wherein the first organ is the subject's liver or pancreas or head of the pancreas, and the second organ is the subject's duodenum or small intestine.

9-16. (canceled)

17. The method of claim 1, wherein during the inserting the dilator assembly is arranged to surround a portion of a guide needle.

18-20. (canceled)

21. The method of claim 1, additionally including hydro-dissecting the first tissue from the second tissue, or the first organ from the second organ, before the hyperechoic marker has reached a target marker-location.

22-24. (canceled)

25. A method for creating a dissected tissue space between a prostate and a rectal wall of a human subject, the method comprising:
a. inserting, through an incision in the subject's perineum, a distal end of a dilator assembly, the dilator assembly arranged to surround a portion of a guiding element penetrating the perineum, the guiding element being one of a guidewire and a guide-needle, the dilator assembly comprising:
i. a distally-beveled dilator, and
ii. a sheath arranged to coaxially surround a portion of the dilator that excludes a distal tip of the dilator, the sheath comprising a hyperechoic marker having an acoustic impedance at least twice that of an outer cylindrical surface portion of the sheath adjacent to the marker, at least a portion of the hyperechoic marker being disposed between 4 and 5 cm from a distal end of the sheath;
b. causing and regulating distal movement of the dilator assembly, the causing and regulating including:
i. responsively to the distal tip of the dilator reaching a first surface of the rectal wall, withdrawing the guiding element, and
ii. responsively to receiving an image from a transrectal ultrasound probe indicating that the hyperechoic marker is parallel to an apex of the prostate, ceasing the distal movement;
c. subsequent to the cessation of the distal movement and while the hyperechoic marker is parallel to the apex of the prostate, (i) withdrawing the dilator, (ii) inserting a deflated tissue-dissection balloon through an interior lumen of the sheath until a distal end of the deflated balloon is aligned with a distal end of the sheath, and (iii) withdrawing the sheath to expose the balloon; and
d. inflating the balloon to cause thereby a separation of the prostate from the rectal wall.

26. The method of claim 25, wherein substantially all of the balloon is deployed beyond the target marker-location.

27. The method of claim 25, wherein a distance from the hyperechoic marker to the distal end of the dilator-sheath matches a length of the balloon

28. The method of claim 27, wherein the distance is from an edge of the hyperechoic marker or from another echogenically distinguishable portion thereof.

29. The method of claim 25, additionally including hydro-dissecting the first tissue from the second tissue, or the first organ from the second organ, before the hyperechoic marker has reached a target marker-location.

30-33. (canceled)

34. A medical dilator kit for an ultrasound-guided tissue dissection, the kit comprising:
a. a beveled-tip medical dilator comprising (i) a conical frustum section formed in a distal portion of the dilator, (ii) a cylinder section formed proximally to the conical frustum portion to abut the base thereof, and
b. a dilator-sheath having an internal diameter greater than or equal to an external diameter of the dilator, the sheath comprising a hyperechoic marker having an acoustic impedance at least twice that of an outer cylindrical surface portion of the dilator-sheath adjacent to the marker, at least a portion of the hyperechoic marker being proximally displaced from a distal end of the dilator-sheath and distally displaced from a proximal end of the dilator-sheath.

35. The dilator kit of claim 34, wherein the at least a portion of the hyperechoic marker is displaced at least 3 cm and not more than 6 cm from the distal end of the dilator-sheath.

36. The dilator kit of claim 35, wherein the at least a portion of the hyperechoic marker is displaced at least 4 cm and not more than 5 cm from the distal end of the dilator-sheath.

37. The dilator kit of claim 34, wherein a distal-tip section of the dilator-sheath includes a beveled circumference.

38. The dilator kit of claim 37, wherein a beveling angle of the distal-tip section of the dilator-sheath is greater than a slant angle of the conical frustrum section of the dilator.

39. The dilator kit of claim 34, wherein the hyperechoic marker is attached to the outer cylindrical surface portion of the dilator-sheath or formed in the outer cylindrical surface portion of the dilator-sheath.

40-42. (canceled)

43. The dilator kit of claim 34, additionally comprising an inter-tissue or inter-organ spacer.

44-51. (canceled)
